(19) United States
(12) Patent Application Publication
(10) Pub. No.: US 2016/0073965 A1
(43) Pub. Date: Mar. 17, 2016

(54) METHODS AND SYSTEMS FOR DETERMINING FLUID RESPONSIVENESS

(71) Applicant: Covidien LP, Mansfield, MA (US)

(72) Inventors: Paul Stanley Addison, Edinburgh (GB); Scott McGonigle, Edinburgh (GB); James Nicholas Watson, Edinburgh (GB); Rui Wang, Livingston (GB); Peter Doyle, Vista, CA (US)

(21) Appl. No.: 14/846,590

(22) Filed: Sep. 4, 2015

Related U.S. Application Data
(60) Provisional application No. 62/051,423, filed on Sep. 17, 2014.

(51) Int. Cl. A61B 5/00; A61B 5/091; A61B 5/08

(52) U.S. Cl. CPC A61B 5/7207; A61B 5/4848; A61B 5/7275; A61B 5/0816; A61B 5/091; A61B 5/7264; A61B 5/725; A61B 5/0059; A61B 5/7246

(57) ABSTRACT

Methods and systems are provided for determining fluid responsiveness based on a physiological signal. The system may determine fluid responsiveness based on the physiological signal and receive or determine respiration information of the subject. The system may correct the fluid responsiveness based on the respiration information. In some embodiments, the system may determine a correction factor to correct the fluid responsiveness values based on a relationship between fluid responsiveness and the respiration information. In some embodiments, the system may correct the measured fluid responsiveness based on an error between the fluid responsiveness measure and another measure such as pulse pressure variation, where there is a relationship between the error and the respiration information.

### SUMMARY

The present disclosure relates to determining fluid responsiveness, and more particularly relates to correcting fluid responsiveness based on respiration information.

Methods and systems are provided for determining fluid responsiveness of a subject. In some embodiments, a physiological signal is received by a system. The system may calculate a fluid responsiveness value based on the physiological signal. The system may determine or receive respiration information. The system may correct the fluid responsiveness value based on the respiration information.

The present disclosure provides embodiments for a physiological monitor for monitoring fluid responsiveness of a subject comprising an input and a fluid responsiveness parameter determination module. The input is configured to receive a physiological signal. The fluid responsiveness parameter determination module is configured to receive the physiological signal, determine a parameter indicative of fluid responsiveness based on the physiological signal, receive respiration information of the subject, determine a correction factor of the parameter indicative of fluid responsiveness based on the respiration information, and determine a corrected parameter indicative of fluid responsiveness based on the parameter indicative of fluid responsiveness and the correction factor.

The present disclosure provides embodiments for a physiological monitor for monitoring fluid responsiveness of a subject comprising a signal generating module, a respiration detection module, and a fluid responsiveness parameter determination module. The signal generating module is configured to generate a physiological signal that is indicative of light attenuated by a subject. The respiration detection module is configured to determine a respiration rate of the subject. The fluid responsiveness parameter determination module is configured to determine a parameter indicative of fluid responsiveness based on respiratory modulations in the physiological signal and the respiration rate of the subject.

The present disclosure provides embodiments for a method of determining fluid responsiveness of a subject comprising receiving a physiological signal and determining respiration information of the subject based on the physiological signal. The method further comprises determining a plurality of amplitudes in the physiological signal and determining a parameter indicative of fluid responsiveness of the subject based on the plurality of amplitudes. The method further comprises calculating a correction factor based on the respiration information and the parameter indicative of fluid responsiveness of the subject and determining a corrected parameter indicative of fluid responsiveness of the subject based on the correction factor and the parameter indicative of fluid responsiveness of the subject.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows a block diagram of an illustrative physiological monitoring system in accordance with some embodiments of the present disclosure;

FIG. 2A shows an illustrative plot of a light drive signal in accordance with some embodiments of the present disclosure;

FIG. 2B shows an illustrative plot of a detector signal that may be generated by a sensor in accordance with some embodiments of the present disclosure;

FIG. 3 is a perspective view of an illustrative physiological monitoring system in accordance with some embodiments of the present disclosure;

FIG. 4 shows an illustrative plot of a PPG waveform reflecting respiratory modulations in accordance with some embodiments of the present disclosure;

FIG. 5 shows an illustrative plot of correcting DPOP based on the relationship between DPOP and Respiration Rate of a given subject;

FIG. 6 shows an illustrative plot of correcting DPOP based on the relationship between DPOP and Respiration Effort of a given subject;

FIG. 7 shows an illustrative plot of correcting DPOP based on the relationship between DPOP and Tidal Volume of a given subject;

FIG. 8 shows an illustrative plot of correcting DPOP based on the relationship between DPOP and Airway Pressure of a given subject;

FIG. 9 shows illustrative steps for determining fluid responsiveness in accordance with some embodiments of the present disclosure; and

FIG. 10 shows an illustrative physiological monitor for monitoring fluid responsiveness in a subject in accordance with some embodiments of the present disclosure.

### DETAILED DESCRIPTION OF THE FIGURES

The present disclosure is directed towards determining fluid responsiveness of a subject. In particular, a monitor is configured to correct a fluid responsiveness value based on respiration information of the subject.

Fluids are commonly delivered to a patient in order to improve the patient's hemodynamic status. Fluid is delivered with the expectation that it will increase the patient's cardiac preload, stroke volume, and cardiac output, resulting in improved oxygen delivery to the organs and tissue. Fluid delivery may also be referred to as volume expansion, fluid therapy, fluid challenge, or fluid loading. However, improved hemodynamic status is not always achieved by fluid loading. Moreover, inappropriate fluid loading may worsen a patient's status, such as by causing hypovolemia to persist (potentially leading to inadequate organ perfusion), or by causing hypervolemia (potentially leading to peripheral or pulmonary edema).

Respiratory variation in the arterial blood pressure waveform is known to be a good predictor of a patient's response to fluid loading, or fluid responsiveness. Fluid responsiveness represents a prediction of whether such fluid loading will improve blood flow within the patient. Fluid responsiveness refers to the response of stroke volume or cardiac output to fluid administration. A patient is said to be fluid responsive if fluid loading does accomplish improved blood flow, such as by an improvement in cardiac output or stroke volume index by about 15% or more. In particular, the pulse pressure variation (PPV) parameter from the arterial blood pressure waveform has been shown to be a good predictor of fluid responsiveness. This parameter can be monitored while adding fluid incrementally, until the PPV value indicates that the patient's fluid responsiveness has decreased, and more fluids will not be beneficial to the patient. This treatment can be accomplished without needing to calculate blood volume or cardiac output directly. This approach, providing incremental therapy until a desired target or endpoint is reached, may be referred to as goal-directed therapy (GDT).

However, determining the PPV is an invasive procedure, requiring the placement of an arterial line in order to obtain the arterial blood pressure waveform. This invasive procedure is time-consuming and presents a risk of infection to the patient. Respiratory variation in a photoplethysmograph (PPG) signal may provide a non-invasive alternative to PPV. The PPG signal can be obtained non-invasively, such as from a pulse oximeter. One measure of respiratory variation in the PPG is the Delta POP metric, which is a measure of the strength of respiratory-induced amplitude modulations of the PPG. This metric assesses changes in the pulse oximetry plethysmograph, and is abbreviated as ΔPOP or DPOP. In addition to DPOP, a number of other measures of respiratory variation may be used to determine fluid responsiveness, including other measures of respiratory-induced amplitude modulations, other respiratory-induced modulations, and any suitable combination thereof. While studies have shown a favorable correlation between DPOP and PPV, there exists a need for more accurate processing of signals to determine DPOP and other similar measures of fluid responsiveness.

Since DPOP is a measure of the strength of respiratory-induced amplitude modulations, it is known to be sensitive to changes in various respiratory parameters or other respiration information of a subject. As used herein, respiratory parameters may be any features or measurements associated with a subject's breathing, such as, for example, respiration rate, respiratory effort, tidal volume, and airway or other respiratory pressure. Thus, there may be situations where such respiratory parameter changes affect the calculation of DPOP even where the patient's fluid responsiveness has not changed. Similarly, the variance of such respiratory parameters between subjects breathing spontaneously and subjects breathing with varying levels of assistance from respiration devices such as ventilators make it difficult to assess the relative fluid responsiveness of subjects accurately. It is thus desirable to estimate or determine the relationship between DPOP and one or more of these respiratory parameters of a subject and correct the calculated DPOP based on the relationship. In accordance with some embodiments of the present disclosure, DPOP may be calculated, respiration information may be determined or otherwise received, and DPOP may be corrected based on the respiration information.

The foregoing techniques may be implemented in an oximeter. An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation invasively by analyzing a blood sample taken from the patient). Pulse oximeters may be included in patient monitoring systems that measure and display various blood flow characteristics including, but not limited to, the blood oxygen saturation (e.g., arterial, venous, or both). Such patient monitoring systems, in accordance with the present disclosure, may also measure and display additional or alternative physiological parameters such as pulse rate, respiration rate, respiration effort, blood pressure, hemoglobin concentration (e.g., oxygenated, deoxygenated, and/or total), systemic vascular resistance, mean arterial pressure, cardiac output, central venous pressure, oxygen demand, adaptive filter parameters, fluid responsiveness parameters, any other suitable physiological parameters, or any combination thereof.

Pulse oximetry may be implemented using a photoplethysmograph. Pulse oximeters and other photoplethysmograph devices may also be used to determine other physiological parameter and information as disclosed in: J. Allen, “Photoplethysmography and its application in clinical physiological measurement.” Physiol. Meas., vol. 28, pp. R1-R39, March 2007; W. B. Murray and P. A. Foster, “The peripheral pulse wave: information overlooked,” J. Clin. Monit., vol. 12, pp. 365-377, September 1996; and K. H. Shelley, “Photoplethysmography: beyond the calculation of arterial oxygen saturation and heart rate,” Anesth. Analg., vol. 105, pp. S31-S36, December 2007; all of which are incorporated by reference herein in their entireties.

An oximeter may include a light sensor that is placed at a site on a patient, typically a fingertip, toe, forehead or earlobe, or in the case of a neonate, across a foot or hand. The oximeter may use a light source to pass light through blood perfused tissue and photoelectrically sense the absorption of the light in the tissue. Additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, around or in front of the ear, and locations with strong pulsatile arterial flow. Suitable sensors for these locations may include sensors that detect reflected light.

The oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, an inverted signal, etc.) may be referred to as the photoplethysmograph (PPG) signal. In addition, the term “PPG signal.” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine arterial blood oxygen saturation using two wavelengths of light and a ratio-of-ratios calculation. As another example, the system may determine regional blood oxygen saturation using two wavelengths of light and two detectors located at different distances from the emitters. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to detect ambient signal levels, reduce overlap of the light drive pulses, allow time for light sources to stabilize, allow time for detected light signals to stabilize or settle, reduce heating effects, reduce power consumption, for any other suitable reason, or any combination thereof.

It will be understood that the techniques described herein are not limited to pulse oximeters and may be applied to any suitable physiological monitoring device.

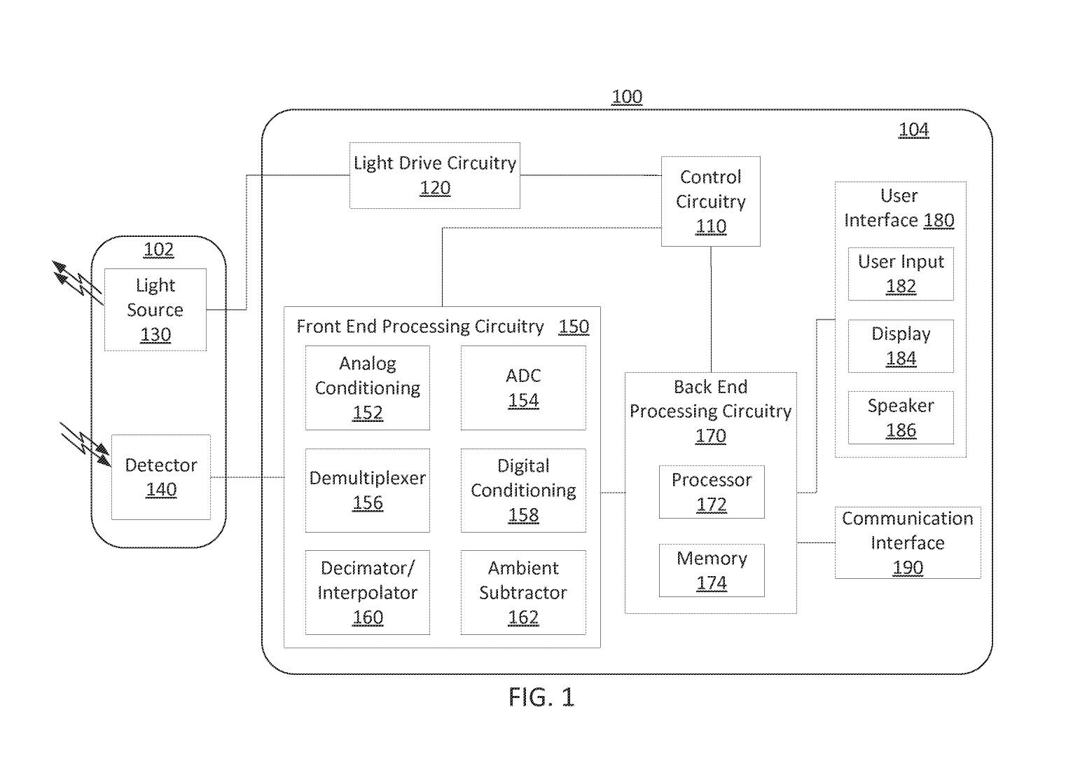

The following description and accompanying FIGS. 1-10 provide additional details and features of some embodiments of the present disclosure.

FIG. 1 shows a block diagram of illustrative physiological monitoring system 100 in accordance with some embodiments of the present disclosure. System 100 may include a sensor 102 and a monitor 104 for generating and processing sensor signals that include physiological information of a subject. In some embodiments, sensor 102 and monitor 104 may be part of an oximeter. In some embodiments, system 100 may include more than one sensor 102.

Sensor 102 of physiological monitoring system 100 may include light source 130 and detector 140. Light source 130 may be configured to emit photonic signals having one or more wavelengths of light (e.g. red and IR) into a subject's tissue. For example, light source 130 may include a red light emitting light source and an IR light emitting light source, e.g. red and IR light emitting diodes (LEDs), for emitting light into the tissue of a subject to generate sensor signals that include physiological information. In one embodiment, the red wavelength may be between about 600 nm and about 750 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detector 140. After converting the received light to an electrical signal, detector 140 may send the detection signal to monitor 104, where the detection signal may be processed and physiological parameters may be determined (e.g., based on the absorption of the red and IR wavelengths in the subject's tissue). In some embodiments, the detection signal may be preprocessed by sensor 102 before being transmitted to monitor 104. Although only one detector 140 is depicted in FIG. 1, in some embodiments, sensor 102 may include additional detectors located at different distances from the light source 130. In embodiments with additional detectors, the sensitivity of the additional detectors may vary based on the distance between the detector and light source 130 such that a far detector may be more sensitive to light than a near detector.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate a light drive signal, which may be used to turn on and off the light source 130, based on the timing control signals. The front end processing circuitry 150 may use the timing control signals to operate synchronously with light drive circuitry 120. For example, front end processing circuitry 150 may synchronize the operation of an analog-to-digital converter and a demultiplexer with the light drive signal based on the timing control signals. In addition, the back end processing circuitry 170 may use the timing control signals to coordinate its operation with front end processing circuitry 150.

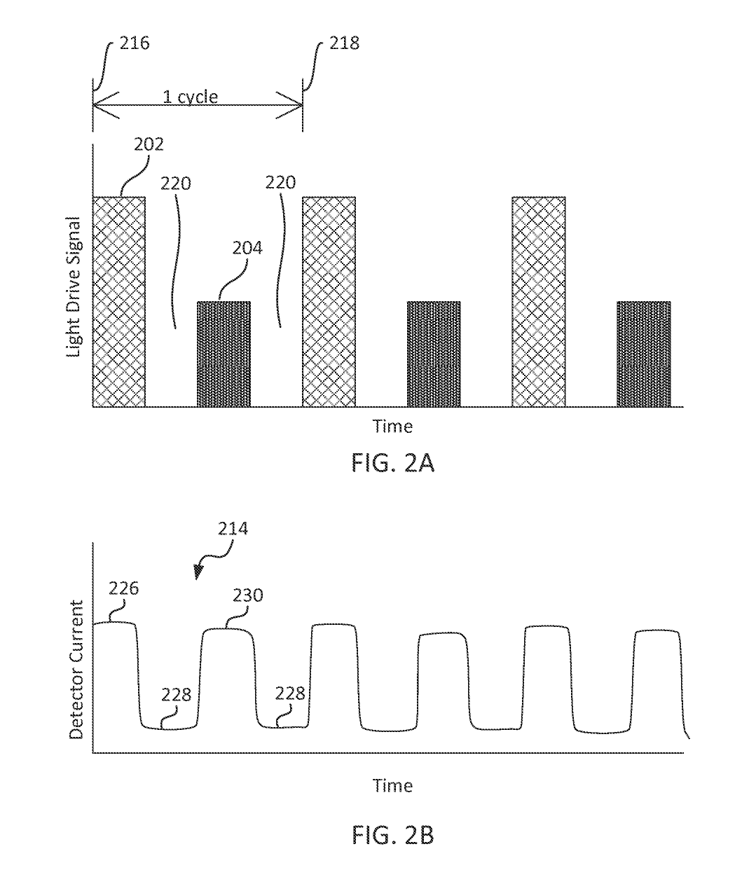

Light drive circuitry 120, as discussed above, may be configured to generate a light drive signal that is provided to light source 130 of sensor 102. The light drive signal may, for example, control the intensity of light source 130 and the timing of when light source 130 is turned on and off. In some embodiments, the intensity of light source 130 may be set based on a gain setting in light drive circuitry 120. When light source 130 is configured to emit two or more wavelengths of light, the light drive signal may be configured to control the operation of each wavelength of light. The light drive signal may comprise a single signal or may comprise multiple signals (e.g., one signal for each wavelength of light). An illustrative light drive signal is shown in FIG. 2A.

In some embodiments, control circuitry 110 and light drive circuitry 120 may generate light drive parameters based on a metric. For example, back end processing 170 may receive information about received light signals, determine light drive parameters based on that information, and send corresponding information to control circuitry 110.

FIG. 2A shows an illustrative plot of a light drive signal including red light drive pulse 202 and IR light drive pulse 204 in accordance with some embodiments of the present disclosure. Light drive pulses 202 and 204 are illustrated as square waves. These pulses may include shaped waveforms rather than a square wave. The shape of the pulses may be generated by a digital signal generator, digital filters, analog filters, any other suitable equipment, or any combination thereof. For example, light drive pulses 202 and 204 may be generated by light drive circuitry 120 under the control of control circuitry 110. As used herein, drive pulses may refer to the high and low states of a shaped pulse, switching power or other components on and off, high and low output states, high and low values within a continuous modulation, other suitable relatively distinct states, or any combination thereof. The light drive signal may be provided to light source 130, including red light drive pulse 202 and IR light drive pulse 204 to drive red and IR light emitters, respectively, within light source 130. Red light drive pulse 202 may have a higher amplitude than IR light drive pulse 204 since red LEDs may be less efficient than IR LEDs at converting electrical energy into light energy. In some embodiments, the output levels may be equal, may be adjusted for nonlinearity of emitters, may be modulated in any other suitable technique, or any combination thereof. Additionally, red light may be absorbed and scattered more than IR light when passing through perfused tissue.

When the red and IR light sources are driven in this manner they emit pulses of light at their respective wavelengths into the tissue of a subject in order to generate sensor signals that include physiological information that physiological monitoring system 100 may process to calculate physiological parameters. It will be understood that the light drive amplitudes of FIG. 2A are merely exemplary and that any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the red and IR light drive pulses. “Off” periods 220 are periods during which no drive current may be applied to light source 130. “Off” periods 220 may be provided, for example, to prevent overlap of the emitted light, since light source 130 may require time to turn completely on and completely off. The period from time 216 to time 218 may be referred to as a drive cycle, which includes four segments: a red light drive pulse 202, followed by an “off” period 220, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red light drive pulse 202 and each IR light drive pulse 204 may be understood to be surrounded by two “off” periods 220. “Off” periods may also be referred to as dark periods, in that the emitters are dark or returning to dark during that period. It will be understood that the particular square pulses illustrated in FIG. 2A are merely exemplary and that any suitable light drive scheme is possible. For example, light drive schemes may include shaped pulses, sinusoidal modulations, time division multiplexing other than as shown, frequency division multiplexing, phase division multiplexing, any other suitable light drive scheme, or any combination thereof.

Referring back to FIG. 1, front end processing circuitry 150 may receive a detection signal from detector 140 and provide one or more processed signals to back end processing circuitry 170. The term “detection signal,” as used herein, may refer to any of the signals generated within front end processing circuitry 150 as it processes the output signal of detector 140. Front end processing circuitry 150 may perform various analog and digital processing of the detector signal. One suitable detector signal that may be received by front end processing circuitry 150 is shown in FIG. 2B.

FIG. 2B shows an illustrative plot of detector current waveform 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current peak 226 may be generated in response to a light source being driven by red light drive pulse 202 of FIG. 2A, and peak 230 may be generated in response to a light source being driven by IR light drive pulse 204. Valleys 228 of detector current waveform 214 may be synchronous with periods of time during which no light is being emitted by the light source, or the light source is returning to dark, such as “off” periods 220. While no light is being emitted by a light source during the valleys, detector current waveform 214 may not fall all of the way to zero.

It will be understood that detector current waveform 214 may be an at least partially idealized representation of a detector signal, assuming perfect light signal generation, transmission, and detection. It will be understood that an actual detector current will include amplitude fluctuations, frequency deviations, droop, overshoot, undershoot, rise time deviations, fall time deviations, other deviations from the ideal, or any combination thereof. It will be understood that the system may shape the drive pulses shown in FIG. 2A in order to make the detector current as similar as possible to idealized detector current waveform 214.

Referring back to FIG. 1, front end processing circuitry 150, which may receive a one or more detection signals, such as detector current waveform 214, may include analog conditioning 152, analog-to-digital converter (ADC) 154, demultiplexer 156, digital conditioning 158, decimator/interpolator 160, and ambient subtractor 162.

Analog conditioning 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some embodiments, one or more gain settings may be used in analog conditioning 152 to adjust the amplification of detector signal.

The conditioned analog signal may be processed by analog-to-digital converter 154, which may convert the conditioned analog signal into a digital signal. Analog-to-digital converter 154 may operate under the control of control circuitry 110. Analog-to-digital converter 154 may use timing control signals from control circuitry 110 to determine when to sample the analog signal. Analog-to-digital converter 154 may be any suitable type of analog-to-digital converter of sufficient resolution to enable a physiological monitor to accurately determine physiological parameters.

Demultiplexer 156 may operate on the analog or digital form of the detector signal to separate out different components of the signal. For example, detector current waveform 214 of FIG. 2B includes a red component corresponding to peak 226, an IR component corresponding to peak 230, and at least one ambient component corresponding to valleys 228. Demultiplexer 156 may operate on detector current waveform 214 of FIG. 2B to generate a red signal, an IR signal, a first ambient signal (e.g., corresponding to the ambient component corresponding to valley 228 that occurs immediately after the peak 226), and a second ambient signal (e.g., corresponding to the ambient component corresponding to valley 228 that occurs immediately after peak 230). Demultiplexer 156 may operate under the control of control circuitry 110. For example, demultiplexer 156 may use timing control signals from control circuitry 110 to identify and separate out the different components of the detector signal.

Digital conditioning 158 may perform any suitable digital conditioning of the detector signal. Digital conditioning 158 may include any type of digital filtering of the signal (e.g., low pass, high pass, band pass, notch, or any other suitable filtering), amplifying, performing an operation on the signal, performing any other suitable digital conditioning, or any combination thereof.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may remove dark or ambient contributions to the received signal or signals.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 154. This may be achieved by applying one or more gains to the detection signal, by analog conditioning 152 to map the expected range of the signal to the full or close to full output range of analog-to-digital converter 154. The output value of analog-to-digital converter 154, as a function of the total analog gain applied to the detection signal, may be given as:

ADC Value=Total Analog Gain×[Ambient Light+LED Light]

Ideally, when ambient light is zero and when the light source is off, the analog-to-digital converter 154 will read just above the minimum input value. When the light source is on, the total analog gain may be set such that the output of analog-to-digital converter 154 may read close to the full scale of analog-to-digital converter 154 without saturating. This may allow the full dynamic range of analog-to-digital converter 154 to be used for representing the detection signal, thereby increasing the resolution of the converted signal. In some embodiments, the total analog gain may be reduced by a small amount so that small changes in the light level incident on the detector do not cause saturation of analog-to-digital converter 154.

However, if the contribution of ambient light is large relative to the contribution of light from a light source, the total analog gain applied to the detection current may need to be reduced to avoid saturating analog-to-digital converter 154. When the analog gain is reduced, the portion of the signal corresponding to the light source may map to a smaller number of analog-to-digital conversion bits. Thus, more ambient light noise in the input of analog-to-digital converter 154 may results in fewer bits of resolution for the portion of the signal from the light source. This may have a detrimental effect on the signal-to-noise ratio of the detection signal. Accordingly, passive or active filtering or signal modification techniques may be employed to reduce the effect of ambient light on the detection signal that is applied to analog-to-digital converter 154, and thereby reduce the contribution of the noise component to the converted digital signal.

Back end processing circuitry 170 may include processor 172 and memory 174. Processor 172 may be adapted to execute software, which may include an operating system and one or more applications, as part of performing the functions described herein. Processor 172 may receive and further process sensor signals received from front end processing circuitry 150. For example, processor 172 may determine one or more physiological parameters based on the received physiological signals. Processor 172 may include an assembly of analog or digital electronic components. Processor 172 may calculate physiological information. For example, processor 172 may compute one or more of fluid responsiveness, a blood oxygen saturation (e.g., arterial, venous, or both), pulse rate, respiration rate, respiration effort, blood pressure, hemoglobin concentration (e.g., oxygenated, deoxygenated, and/or total), any other suitable physiological parameters, or any combination thereof. Processor 172 may perform any suitable signal processing of a signal, such as any suitable scaling, band-pass filtering, adaptive filtering, closed-loop filtering, any other suitable filtering, and/or any combination thereof. Processor 172 may also receive input signals from additional sources not shown. For example, processor 172 may receive an input signal containing information about treatments provided to the subject from user interface 180. Additional input signals may be used by processor 172 in any of the calculations or operations it performs in accordance with back end processing circuitry 170 or monitor 104.

Memory 174 may include any suitable computer-readable media capable of storing information that can be interpreted by processor 172. In some embodiments, memory 174 may store calculated values, such as pulse rate, blood pressure, blood oxygen saturation, fiducial point locations or characteristics, initialization parameters, systemic vascular resistance, mean arterial pressure, cardiac output, central venous pressure, oxygen demand, adaptive filter parameters, fluid responsiveness parameters, any other calculated values, or any combination thereof, in a memory device for later retrieval. This information may be data or may take the form of computer-executable instructions, such as software applications, that cause a microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled with user interface 180 and communication interface 190.

User interface 180 may include user input 182, display 184, and speaker 186. User interface 180 may include, for example, any suitable device such as one or more medical devices (e.g., a medical monitor that displays various physiological parameters, a medical alarm, or any other suitable medical device that either displays physiological parameters or uses the output of back end processing 170 as an input), one or more display devices (e.g., monitor, personal digital assistant (PDA), mobile phone, tablet computer, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth.

In an embodiment, the subject may be a medical patient and display 184 may exhibit a list of values which may generally apply to the patient, such as, for example, age ranges or medication families, which the user may select using user input 182. Additionally, display 184 may display, for example, an estimate of a subject's blood oxygen saturation generated by monitor 104 (e.g., an “SpO₂” or a regional oximetry measurement), fluid responsiveness information, pulse rate information, respiration rate and/or effort information, blood pressure information, hemoglobin concentration information, systemic vascular resistance, mean arterial pressure, cardiac output, central venous pressure, oxygen demand, any other parameters, and any combination thereof. Display 184 may include any type of display such as a cathode ray tube display, a flat panel display such as a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof.

Communications interface 190 may be configured to allow wired communication (e.g., using USB, RS-232, Ethernet, or other standards), wireless communication (e.g., using WiFi, IR, WiMax, BLUETOOTH, USB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable.

In some embodiments, communications interface 190 may include an internal bus such as, for example, one or more slots for insertion of expansion cards. In some embodiments, communications interface 190 may enable monitor 104 to exchange information with external devices such as a ventilator, a capnograph, a trans-thoracic impedance device, a pneumotachometer, any other suitable external devices, and any combination thereof. For example, communications interface 190 may receive respiration information from any of the above-mentioned external devices, any other suitable devices, or any suitable combination thereof.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In other embodiments, the functionality of one or more of the components may be performed in a different order or may not be required. In an embodiment, all of the components of physiological monitoring system 100 can be realized in processor circuitry.

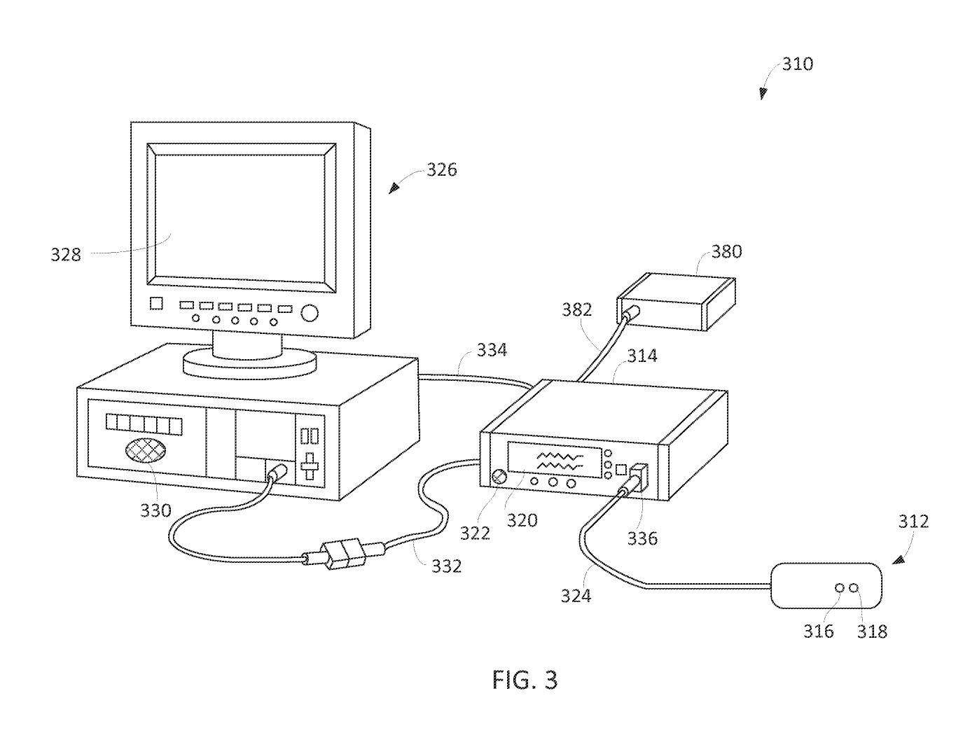

FIG. 3 is a perspective view of an illustrative physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. One or more detector 318 may also be provided in sensor unit 312 for detecting the light that is reflected by or has traveled through the subject's tissue. Any suitable configuration of light source 316 and detector 318 may be used. In an embodiment, sensor unit 312 may include multiple light sources and detectors, which may be spaced apart. Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two different locations on a subject's body. In an example, an oximeter sensor may be located at a first position and a thermodilution sensor may be located at a second location. In another example, an oximeter sensor and a temperature sensor may be located near to one another or in the same structure.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected (not shown) to monitor 314. Monitor 314 may be configured to calculate physiological parameters based at least in part on data relating to light emission and light detection received from one or more sensor units such as sensor unit 312. For example, monitor 314 may be configured to determine fluid responsiveness, pulse rate, respiration rate, respiration effort, blood pressure, blood oxygen saturation (e.g., arterial, venous, regional, or a combination thereof), hemoglobin concentration (e.g., oxygenated, deoxygenated, and/or total), systemic vascular resistance, mean arterial pressure, cardiac output, central venous pressure, oxygen demand, any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range. In some embodiments, physiological monitoring system 310 may include a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324 at port 336. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The monitor 326 may include a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide a display 328 for information from monitor 314 and from other medical monitoring devices or systems (not shown). For example, multi-parameter physiological monitor 326 may be configured to display an estimate of a subject's fluid responsiveness, blood oxygen saturation, and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

In some embodiments, any of the processing components and/or circuits, or portions thereof, of FIGS. 1 and 3, including sensors 102 and 312 and monitors 104, 314, and 326, may be referred to collectively as processing equipment. For example, processing equipment may be configured to amplify, filter, sample and digitize an input signal from sensor 102 or 312 (e.g., using an analog-to-digital converter), and calculate physiological information from the digitized signal. Processing equipment may be configured to generate light drive signals, amplify, filter, sample and digitize detector signals, sample and digitize other analog signals, calculate physiological information from the digitized signal, perform any other suitable processing, or any combination thereof. In some embodiments, all or some of the components of the processing equipment may be referred to as a processing module.

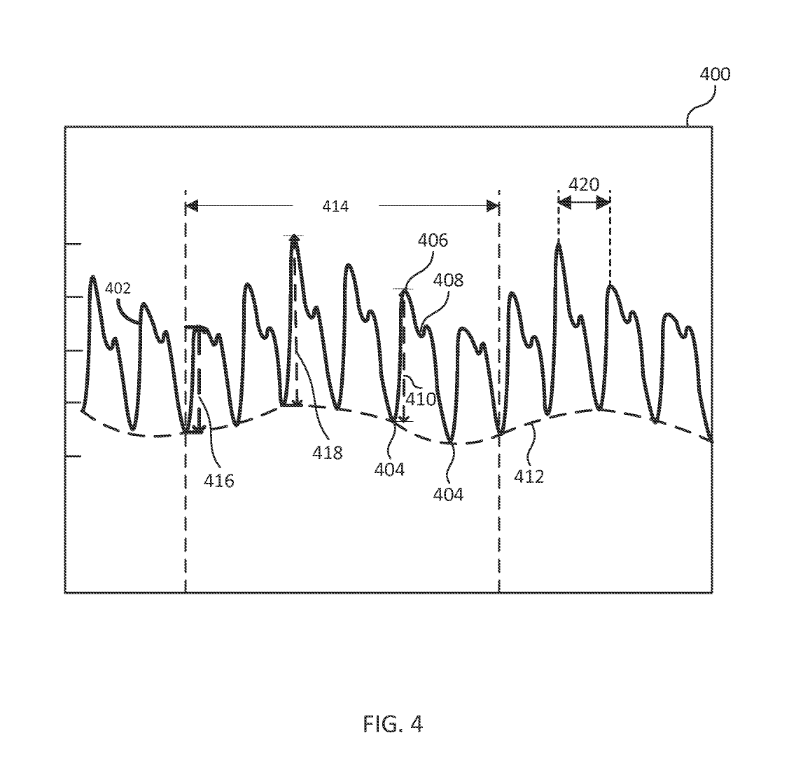

As described above, respiratory variation in the arterial blood pressure waveform is known to be a good predictor of a subject's fluid responsiveness. In particular, the PPV of a subject is known to be a good predictor of fluid responsiveness, but, as described above, requires invasive procedures to determine. Accordingly, determining respiratory variation in a PPG signal from a pulse oximeter may provide a non-invasive alternative to determining the PPV of a subject. Determination of fluid responsiveness in accordance with the present disclosure will be discussed with reference to FIGS. 4-10 below. Although a PPG signal from a pulse oximeter is used to illustrate embodiments of the present disclosure, it will be understood that the techniques described herein are not limited to PPG signals and pulse oximeters and may be applied to any suitable physiological signals and monitoring devices.

FIG. 4 shows an illustrative plot 400 of PPG waveform 402 reflecting respiratory modulations in accordance with some embodiments of the present disclosure. PPG waveform 402 may be generated, for example, by system 100 of FIG. 1 or system 310 of FIG. 3. As illustrated, PPG waveform 402 represents the absorption of light by a subject's tissue over time. PPG waveform 402 includes pulses where the absorption of light increases due to the increased volume of blood in the arterial blood vessel due to cardiac pulses. In some embodiments, pulses may be identified between adjacent valleys 404 and as illustrated may include a peak 406 and a dichrotic notch 408. The pulses include an upstroke between the first valley and the main peak. For example, an upstroke is depicted in FIG. 4 between the first valley 404 and peak 406. The amplitude of this upstroke is depicted as amplitude 410 measured from the first valley 404 to peak 406. Other amplitude values may be derived from the PPG waveform, such as a downstroke amplitude, average amplitude, or area under the pulse. In some embodiments, the amplitude of a pulse may be determined by subtracting a minimum value of PPG waveform 402 from a maximum value of PPG waveform 402 within a segment of PPG waveform 402 that generally corresponds to the period of a pulse. PPG waveform 402 also includes a varying baseline 412. PPG waveform 402 modulates above baseline 412 due to the pulses.

For most subjects, the PPG signal is affected by the subject's respiration, i.e. inhaling and exhaling, resulting in certain respiration modulations in the PPG waveform. FIG. 4 illustrates respiration modulations in PPG waveform 402 as a result of the subject's inhaling and exhaling. One type of respiratory modulation is the modulation of baseline 412 of PPG waveform 402. The effect of the subject's breathing in and out causes the baseline of the waveform 402 to move up and down, cyclically, with the subject's respiration. The baseline may be tracked by following any fiducial of PPG waveform 402, such as the peaks 406, valleys 404, dichrotic notches 408, median value, or any other fiducials. A second type of respiration-induced modulation of PPG waveform 402 is the modulation of pulse amplitudes. As the patient breathes in and out, the amplitudes of pulses decreases and increases, with larger amplitudes tending to coincide with the top of the baseline shift, and smaller amplitudes tending to coincide with the bottom of the baseline shift (though the larger and smaller amplitudes do not necessarily fall at the top and bottom of the baseline shift). A third respiratory type of modulation is the modulation of period 420 between pulses (also referred to as frequency modulation). Each of these modulations may be referred to as a respiratory component of PPG waveform 402, or a respiratory-induced modulation of PPG waveform 402. It should be noted that a particular individual may exhibit only the baseline modulation, or only the amplitude modulation, or only the frequency modulation, or any combination thereof. As referred to herein, a respiratory component of the PPG waveform 402 includes any one of these respiratory-induced modulations of PPG waveform 402, a measure of these modulations, or a combination of them.

The respiratory modulations of PPG waveform 402 can be affected by a subject's fluid status. For example, a hypovolemic subject may exhibit relatively larger respiratory variations of PPG waveform 402. When a subject loses fluid, the subject may have decreased cardiac output or stroke volume, which tends to increase the respiratory variations present in the subject's PPG waveform. Specifically, the baseline modulation, amplitude modulation, and frequency modulation may become more pronounced. Thus, larger respiratory modulations may indicate that the subject will respond favorably to fluid loading, whereas smaller respiratory modulations may indicate that a patient may not respond favorably to fluid loading. The respiratory modulations of the PPG waveform 402 may be identified and used to determine a subject's fluid responsiveness.

In some embodiments, a physiological monitor receives a PPG signal and determines a parameter indicative of fluid responsiveness based on the PPG signal. In some embodiments, the parameter indicative of fluid responsiveness is a measure of a subject's likely response to fluid therapy. In some embodiments, the parameter indicative of fluid responsiveness is a metric that reflects a degree of respiratory variation of the PPG signal. One example of a parameter indicative of fluid responsiveness is a measure of the amplitude modulations of the PPG signal, such as Delta POP (DPOP or ΔPOP, defined below). Another example of a parameter indicative of fluid responsiveness is a measure of the baseline modulation of the PPG. In some embodiments, other suitable metrics or combinations of metrics may be used to assess the respiratory modulation of the PPG signal. For example, a parameter indicative of fluid responsiveness may be based on the amplitudes or areas of acceptable pulses within a particular time frame or window. For example, as illustrated in FIG. 4, minimum amplitude 416 of the pulses within respiratory period 414 may be subtracted from maximum amplitude 418 within respiratory period 414 and then divided by an average or mean value of minimum amplitude 416 and maximum amplitude 418. In some embodiments, a parameter indicative of fluid responsiveness may be derived from the period or frequency of pulses within a time frame or window. For example, a modulation or variation in the period or frequency among two or more cardiac pulses may be used to derive a parameter indicative of fluid responsiveness. In general, the parameter indicative of fluid responsiveness may be based on one or more respiratory variations exhibited by the PPG waveform 402. Further, a parameter indicative of fluid responsiveness may be determined through the use of wavelet transforms, such as described in United States Patent Application Publication No. 2010/0324827, entitled “Fluid Responsiveness Measure,” which is hereby incorporated by reference in its entirety.

In some embodiments, DPOP is used as the parameter indicative of fluid responsiveness. The DPOP metric can be calculated from PPG waveform 402 for a particular time window as follows:

DPOP=(AMPₘₐₓ−AMPₘᵢₙ)/AMPₐᵥₑ (1)

where AMPₘₐₓ represents the maximum amplitude (such as maximum amplitude 418 in FIG. 4) during a time window (such as respiratory period 414 in FIG. 4), AMPₘᵢₙ represents the minimum amplitude (such as minimum amplitude 416 in FIG. 4) during the time window, and AMPₐᵥₑ is the average of the two, as follows:

AMPₐᵥₑ=(AMPₘₐₓ+AMPₘᵢₙ)/2 (2)

In some embodiments, AMPₘₐₓ and AMPₘᵢₙ may be measured at other locations of the PPG, such as within or along a pulse. DPOP is a measure of the respiratory variation in the AC portion of the PPG signal. DPOP is a unit-less value, and in some embodiments can be expressed as a percentage. In some embodiments, respiratory period 414 is one respiratory cycle (inhalation and exhalation). In some embodiments, respiratory period 414 is a fixed duration of time that approximates one respiratory cycle, such as 5 seconds, 10 seconds, or any other suitable duration. In some embodiments, respiratory period 414 may be adjusted dynamically based on the subject's calculated or measured respiration rate, so that the period is approximately the same as one respiratory cycle period. In some embodiments, a signal turning point detector may be used to identify the maximum and minimum points in the PPG signal, in order to calculate the upstroke amplitudes.

In some embodiments, it is desirable to determine the parameter indicative of fluid responsiveness by averaging the parameter as calculated in accordance with any of the embodiments described above over a second time window. For example, if DPOP is used as the parameter indicative of fluid responsiveness, and is calculated over a fixed duration of 10 seconds, it may be desirable to average the plurality of DPOP calculations performed over a fixed window of 120 seconds, effectively taking the average of 12 DPOP calculations to yield a parameter indicative of the subject's fluid responsiveness.

Fluid responsiveness parameters such as DPOP determined based on respiratory modulations as described above may depend on respiratory parameters of the subject, such as, for example, the respiration rate (RR), the respiration effort (RE), the tidal volume (TV), the airway or other respiratory pressure, any other respiration information of the subject, or any suitable combination thereof. Accordingly, changes in such respiratory parameters may cause a change in DPOP (or other fluid responsiveness parameters based on respiratory modulations) that decreases the accuracy of the DPOP value with respect to PPV, and in turn, or alternatively, with respect to the subject's actual fluid responsiveness. It is therefore desirable to adjust the determination of the subject's fluid responsiveness accordingly.

As described above, a subject's DPOP (or other fluid responsiveness parameters based on respiratory modulations) as calculated above may depend on the RR of a subject. Specifically, as the RR increases, there is less time for the underlying hemodynamic system to respond, such that the modulations of the plethysmograph waveform may reduce in magnitude. Since DPOP depends on the modulations of the plethysmograph waveform, the reduction in plethysmograph modulation causes a reduction of the value of DPOP. As described above, however, such reduction in the DPOP value may not accurately reflect a change in the fluid responsiveness of the subject. In some embodiments, in order to correct the DPOP to more accurately reflect the fluid responsiveness of the subject, the relationship between DPOP and the RR may be determined. In some embodiments, a measured DPOP value may be corrected by determining the DPOP value that would have been measured at a standard RR, based on the determined relationship between DPOP and RR.

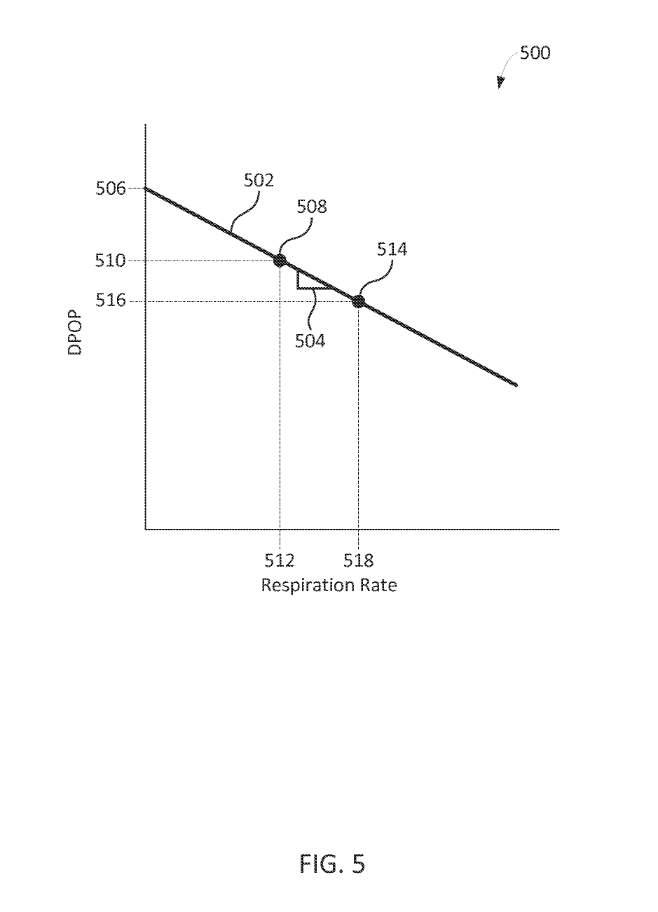

An exemplary illustration of correcting DPOP based on the relationship between DPOP and RR of a given subject is illustrated in plot 500 of FIG. 5. In some embodiments, the nature of the relationship between DPOP and RR may be determined empirically based on measured data, by trained neural networks, or by any other suitable method. In some embodiments, the mathematical relationship between DPOP and RR may be expressed by a linear equation, a polynomial equation, or any suitable equation. For example, FIG. 5 illustrates an embodiment in which it is determined that there is a linear relationship between DPOP and the RR. Specifically, this linear relationship is illustrated by line 502, which has a slope 504 and a vertical axis crossing point 506. As described above, line 502 indicates that the value of DPOP decreases as the RR of the subject increases. In the case of a linear relationship between DPOP and RR, the relationship may be expressed by the equation:

DPOP=m*RR+A, (3)

where m is the slope (e.g., 504 in FIG. 5) of the line and A is the vertical axis crossing point (e.g., 506 in FIG. 5). In some embodiments, once the relationship between DPOP and RR is known, a linear correction may be applied to a measured DPOP (denoted DPOP_{M}) based on a measured RR (denoted RR_{M}) and a standard, or normalizing value of RR (denoted RR) by the following equation:

DPOP_{C}=DPOP_{M}−(RR_{M}−RR_{C})*m, (4)

where DPOP_{C} is the corrected DPOP value. It will be understood that the portion of eq. (4) above used to correct the measured DPOP_{M}, in this case (RR_{M}−RR_{C})*m, may be referred to as a correction factor.

In some embodiments, eq. (4) above may be used with reference to FIG. 5 to correct a measured DPOP value. For example, point 508 may represent a measured DPOP 510 and a measured RR 512, and RR 518 may represent the standard RR of a subject. In some embodiments, the standard RR may be an RR typically used for a subject who is being ventilated. For example, the standard RR may be 12 breaths per minute. In some embodiments, when measured RR 512 does not correspond to standard RR 518, it may be desirable to correct measured DPOP 510 by calculating the DPOP that would have been measured if the RR was at the standard RR 518. Accordingly, in some embodiments, a corrected DPOP 516 may be determined based on 510, 512, 518, 504, and Eq. 4 above.

It will be understood that while the discussion above and FIG. 5 both refer to a linear relationship between DPOP and RR, a non-linear relationship between DPOP and RR may be used in some embodiments to correct DPOP. For example, in some embodiments it may be determined that the relationship between DPOP and RR can be expressed by the polynomial equation:

DPOP=m₁*RR²+m₂*RR+A (5)

In such a case, a non-linear correction may be applied using the following equation:

DPOP_{C}=DPOP_{M}−(RR_{M}²−RR_{C}²)*m₁−(RR_{M}−RR_{C})*m₂ (6)

It will be understood that in the case of eq. (6), (RR_{M}²−RR_{C}²)*m₁+(RR_{M}−RR_{C})*m₂, may be referred to as a correction factor. It will also be understood that in some embodiments, a higher order polynomial equation or any other suitable non-linear equation may be used to express the relationship between DPOP and RR, and appropriate correction may be applied based on the equation, the measured DPOP value, the measured RR value, and the standard RR value similar to that described above. For example, any suitable non-parametric and/or heteroassociative mapping may be used to create corrective formulas as alternatives to Eqs. (4) and (6) above, each with different correction factors used to correct the measured DPOP value.

As described above, a subject's calculated DPOP (or other calculated fluid responsiveness parameters based on respiratory modulations) may depend on the Respiratory Effort (RE) of a subject. As used herein, RE refers to the effort associated with a subject's respiration. For example, RE may be a measure of the effort required to breath. As opposed to the relationship between RR and DPOP, it may be determined that an increase in RE may result in an increase in DPOP. This is because increased respiratory effort of a subject may lead to increased respiratory modulations, which in turn lead to an increase in the DPOP value. Similar to the relationship between RR and DPOP, a change in the DPOP value based on a change in RE may not accurately reflect the fluid responsiveness of the subject. Accordingly, in some embodiments, in order to correct the DPOP to more accurately reflect the fluid responsiveness of the subject, the relationship between DPOP and the RE may be determined. In some embodiments, a measured DPOP value may be corrected by determining the DPOP value that would have been measured at a standard RE, based on the determined relationship between DPOP and RE.

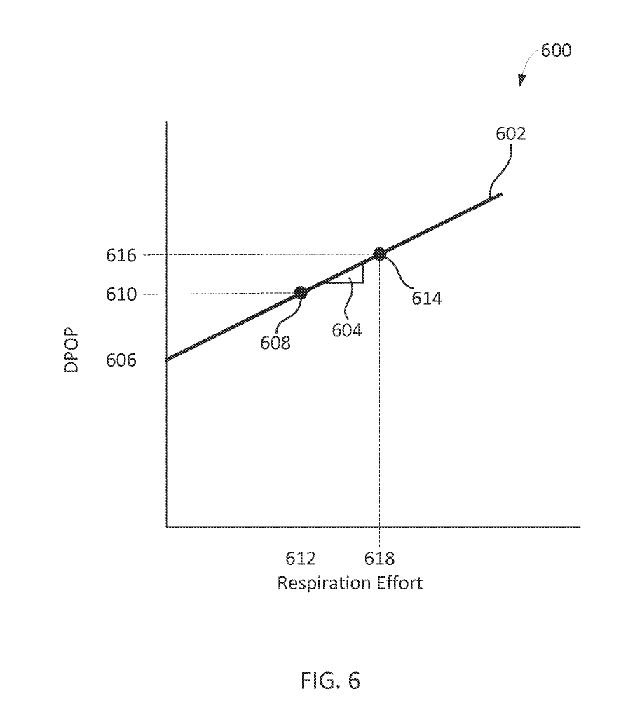

An exemplary illustration of correcting DPOP based on the relationship between DPOP and RE of a given subject is illustrated in plot 600 of FIG. 6. Similar to that of DPOP and RR, in some embodiments, the nature of the relationship between DPOP and RE may be determined empirically based on measured data, by trained neural networks, or by any other suitable method. In some embodiments, the mathematical relationship between DPOP and RE may be expressed by a linear equation, a polynomial equation, or any suitable equation. For example, FIG. 6 illustrates an embodiment in which it is determined that there is a linear relationship between DPOP and the RE. Specifically, this linear relationship is illustrated by line 602, which has a slope 604 and a vertical axis crossing point 606. As described above, line 602 indicates that the value of DPOP increases as the RE of the subject increases. In the case of a linear relationship between DPOP and RE, the relationship may be expressed by the equation:

DPOP=m*RE+A, (7)

where m is the slope (e.g., 604 in FIG. 6) of the line and A is the vertical axis crossing point (e.g., 606 in FIG. 6). In some embodiments, once the relationship between DPOP and RE is known, a linear correction may be applied to a measured DPOP (denoted DPOP_{M}) based on a measured RE (denoted RE_{M}) and a standard, or normalizing value of RE (denoted RE_{C}) by the following equation:

DPOP_{C}=DPOP_{M}−(RE_{M}−RE_{C})*m, (8)

where DPOP_{C} is the corrected DPOP value. It will be understood that, similar to eq. (4) above, the portion of eq. (8) above used to correct the measured DPOP_{M}, in this case (RE_{M}−RE_{C})*m, may be referred to as a correction factor.

In some embodiments, eq. (8) above may be used with reference to FIG. 6 to correct a measured DPOP value. For example, point 608 may represent a measured DPOP 610 and a measured RE 612, and RE 618 may represent a standard RE of a subject. In some embodiments, the standard RE may represent the typical amount of effort required to breathe when there is no airway obstruction in a subject and/or when the subject has a normal level of lung/chest wall compliance. In some embodiments, when measured RE 612 does not correspond to standard RE 618, it may be desirable to correct measured DPOP 610 by calculating the DPOP that would have been measured if the RE was at the standard RE 618. Accordingly, in some embodiments, a corrected DPOP 616 may be determined based on 610, 612, 618, 604, and Eq. (8) above.

It will be understood that while the discussion above and FIG. 6 both refer to a linear relationship between DPOP and RE, the present disclosure is also applicable to correct DPOP in the case of a non-linear relationship between DPOP and RE. For example, in some embodiments it may be determined that the relationship between DPOP and RE can be expressed by the polynomial equation:

DPOP=m₁*RE²+m₂*RE+A (9)

In such a case, a non-linear correction may be applied using the following equation:

DPOP_{C}=DPOP_{M}−(RE_{M}²−RE²)*m₁−(RE_{M}−RE_{C})*m₂ (10)

It will be understood that in the case of eq. (10), (RE_{M}²−RE_{C}²)*m₁+(RE_{M}−RE_{C})*m₂, may be referred to as a correction factor. It will also be understood that in some embodiments, a higher order polynomial equation or any other suitable non-linear equation may be used to express the relationship between DPOP and RE, and appropriate correction may be applied based on the equation, the measured DPOP value, the measured RE value, and the standard RE value similar to that described above. For example, any suitable non-parametric and/or heteroassociative mapping may be used to create corrective formulas as alternatives to Eqs. (8) and (10) above, each with different correction factors used to correct the measured DPOP value.

As described above, a subject's calculated DPOP (or other calculated fluid responsiveness parameters based on respiratory modulations) may depend on the Tidal Volume (TV) of a subject. As used herein, TV refers to the volume of air inspired or expired in a single breath. As opposed to the relationship between RR and DPOP, and similar to the relationship between RE and DPOP, it may be determined that an increase in TV may result in an increase in DPOP. This is because increased TV of a subject leads to increased respiratory modulations, which in turn leads to an increase in the DPOP value of the subject. Similar to the relationship between RR and DPOP and RE and DPOP, a change in the DPOP value based on a change in TV may not accurately reflect a change in the fluid responsiveness of the subject. Accordingly, in some embodiments, in order to correct the DPOP to more accurately reflect the fluid responsiveness of the subject, the relationship between DPOP and the TV may be determined. In some embodiments, a measured DPOP value may be corrected by determining the DPOP value that would have been measured at a standard TV, based on the determined relationship between DPOP and TV.

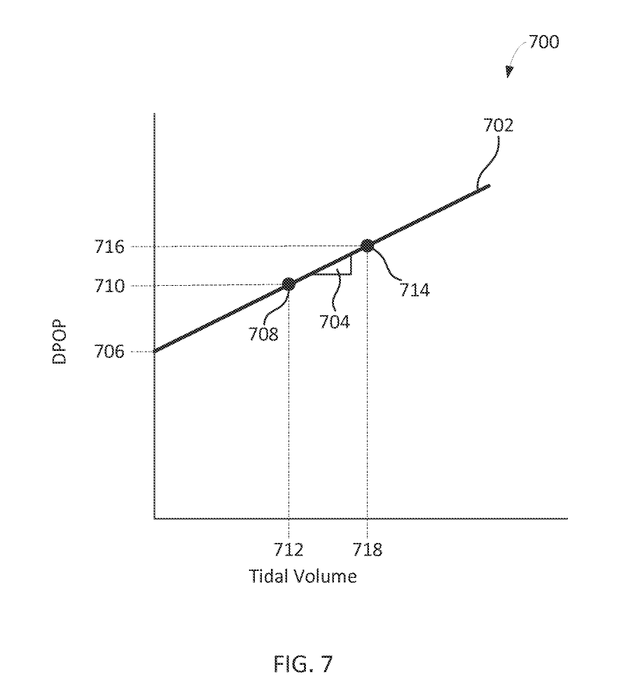

An exemplary illustration of correcting DPOP based on the relationship between DPOP and TV of a given subject is illustrated in plot 700 of FIG. 7. Similar to that of DPOP and RR, in some embodiments, the nature of the relationship between DPOP and TV may be determined empirically based on measured data, by trained neural networks, or by any other suitable method. In some embodiments, the mathematical relationship between DPOP and TV may be expressed by a linear equation, a polynomial equation, or any suitable equation. For example, FIG. 7 illustrates an embodiment in which it is determined that there is a linear relationship between DPOP and the TV. Specifically, this linear relationship is illustrated by line 702, which has a slope 704 and a vertical axis crossing point 706. As described above, line 702 indicates that the value of DPOP increases as the TV of the subject increases. In the case of a linear relationship between DPOP and TV, the relationship may be expressed by the equation:

DPOP=m*TV+A, (11)

where m is the slope (e.g., 704 in FIG. 7) of the line and A is the vertical axis crossing point (e.g., 706 in FIG. 7). In some embodiments, once the relationship between DPOP and TV is known, a linear correction may be applied to a measured DPOP (denoted DPOP_{M}) based on a measured TV (denoted TV_{M}) and a standard, or normalizing value of TV (denoted TV_{C}) by the following equation:

DPOP_{C}=DPOP_{M}−(TV_{M}−TV_{C})*m, (12)

where DPOP_{C} is the corrected DPOP value. It will be understood that, similar to eqs. (4) and (8) above, the portion of eq. (12) above used to correct the measured DPOP_{M}, in this case (TV_{M}−TV_{C})*m, may be referred to as a correction factor.

In some embodiments, eq. (12) above may be used with reference to FIG. 7 to correct a measured DPOP value. For example, point 708 may represent a measured DPOP 710 and a measured TV 712, and TV 718 may represent a standard TV of a subject. In some embodiments, the standard TV may be a TV typical of a subject who is being ventilated. For example, the standard TV may be obtained from an average value across a population of subjects. In some embodiments, the standard TV may be 6 ml/Kg of predicted body weight. In some embodiments, when measured TV 712 does not correspond to standard TV 718, it may be desirable to correct measured DPOP 710 by calculating the DPOP that would have been measured if the TV was at the standard TV 718. Accordingly, in some embodiments, a corrected DPOP 716 may be determined based on 710, 712, 718, 704, and Eq. (12) above.

It will be understood that while the discussion above and FIG. 7 both refer to a linear relationship between DPOP and TV, a non-linear relationship between DPOP and TV may be used in some embodiments to correct DPOP. For example, in some embodiments it may be determined that the relationship between DPOP and TV can be expressed by the polynomial equation:

DPOP=m₁*TV²+m₂*TV+A (13)

In such a case, a non-linear correction may be applied using the following equation:

DPOP_{C}=DPOP_{M}−(TV_{M}²−TV_{C}²)*m₁−(TV_{M}−TV_{C})*m₂ (14)

It will be understood that in the case of eq. (14), (TV_{M}−TV_{C}²)*m₁+(TV_{M}−TV_{C})*m₂, may be referred to as a correction factor. It will also be understood that in some embodiments, a higher order polynomial equation or any other suitable non-linear equation may be used to express the relationship between DPOP and TV, and appropriate correction may be applied based on the equation, the measured DPOP value, the measured TV value, and the standard TV value similar to that described above. For example, any suitable non-parametric and/or heteroassociative mapping may be used to create corrective formulas as alternatives to Eqs. (12) and (14) above, each with different correction factors used to correct the measured DPOP value.

As described above, a subject's calculated DPOP (or other calculated fluid responsiveness parameters based on respiratory modulations) may depend on certain pressures within the airway or respiratory system of a subject (referred to herein as “PA” for ease of reference). As used herein, PA refers to either measured or estimated pressure in the airway of a subject, the pleural space of the subject, or across the respiratory system of the subject, or any parameters indicative thereof. For example, PA may include the upper airway pressure, the carinal pressure, the transthoracic pressure, the transpulmonary pressure, the pleural pressure, and/or any suitable pressure value or estimate. Any of the above or similar pressure measurements or estimates may be obtained in any suitable way as is known in the art. In some embodiments, airway resistance may be calculated to obtain a better estimate of carinal pressure. For example, a ventilator may calculate the airway resistance or may use a look-up table to estimate the airway resistance using the specifications of the artificial airway such as the internal diameter and estimated or measured lung flow. In some embodiments, a ventilator may obtain a plateau measurement at the end of a subject's inspiration when flow is at or near zero to provide a better lung pressure estimate. In some embodiments, the pressure value may be made more accurate by taking into account the resistance caused by a pulmonary disease, such as asthma. PA of a given subject may generally vary over a respiratory cycle, so in some embodiments, PA may refer to a suitable reference PA during the respiratory cycle. For example, PA may refer to a maximum PA within a respiratory cycle. As opposed to the relationship between RR and DPOP, and similar to the relationship between RE and DPOP, and TV and DPOP, it may be determined that an increase in PA may result in an increase in DPOP. This is because increased PA of a subject leads to increased respiratory modulations, which in turn leads to an increase in the DPOP value of the subject. Similar to the relationship between RR and DPOP, RE and DPOP, and TV and DPOP, a change in the DPOP value based on a change in PA may not accurately reflect the fluid responsiveness of the subject. Accordingly, in some embodiments, in order to correct the DPOP to more accurately reflect the fluid responsiveness of the subject, the relationship between DPOP and the PA may be determined. In some embodiments, a measured DPOP value may be corrected by determining the DPOP value that would have been measured at a standard PA, based on the determined relationship between DPOP and PA.

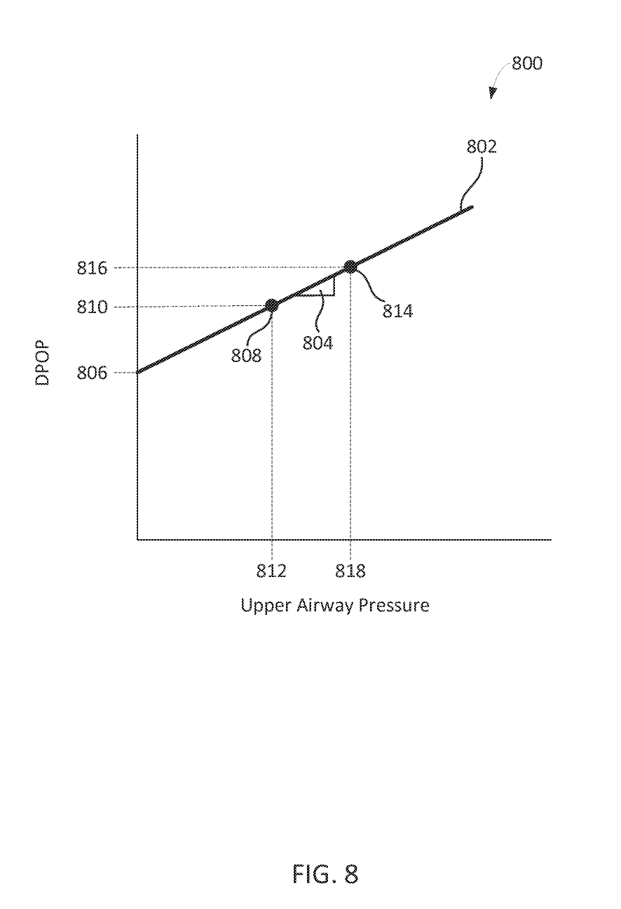

An exemplary illustration of correcting DPOP based on the relationship between DPOP and PA of a given subject is illustrated in plot 800 of FIG. 8. Similar to that of DPOP and RR, in some embodiments, the nature of the relationship between DPOP and PA may be determined empirically based on measured data, by trained neural networks, or by any other suitable method. In some embodiments, the mathematical relationship between DPOP and PA may be expressed by a linear equation, a polynomial equation, or any suitable equation. For example, FIG. 8 illustrates an embodiment in which it is determined that there is a linear relationship between DPOP and the PA. Specifically, this linear relationship is illustrated by line 802, which has a slope 804 and a vertical axis crossing point 806. As described above, line 802 indicates that the value of DPOP increases as the PA of the subject increases. In the case of a linear relationship between DPOP and PA, the relationship may be expressed by the equation:

DPOP=m*PA+A, (15)

where m is the slope (e.g., 804 in FIG. 8) of the line and A is the vertical axis crossing point (e.g., 806 in FIG. 8). In some embodiments, once the relationship between DPOP and PA is known, a linear correction may be applied to a measured DPOP (denoted DPOP_{M}) based on a measured PA (denoted PA_{M}) and a standard, or normalizing value of PA (denoted PA_{C}) by the following equation:

DPOP_{C}=DPOP_{M}−(PA_{M}−PA_{C})*m, (16)

where DPOP_{C} is the corrected DPOP value. It will be understood that, similar to eqs. (4), (8), and (12) above, the portion of eq. (16) above used to correct the measured DPOP_{M}, in this case (PA_{M}−PA_{C})*m, may be referred to as a correction factor.

In some embodiments, eq. (16) above may be used with reference to FIG. 8 to correct a measured DPOP value. For example, point 808 may represent a measured DPOP 810 and a measured PA 812, and PA 818 may represent a standard PA of a subject. In some embodiments, the standard PA may be a PA typical of a subject who is being ventilated. For example, if maximum airway pressure is used as the reference PA, the standard PA may be 1-2 cmH₂O, and if the maximum pleural pressure is used, the standard PA may be 3-5 cmH₂O. In some embodiments, when measured PA 812 does not correspond to standard PA 818, it may be desirable to correct measured DPOP 810 by calculating the DPOP that would have been measured if the PA was at the standard PA 818. Accordingly, in some embodiments, a corrected DPOP 816 may be determined based on 810, 812, 818, 804, and Eq. (16) above.

It will be understood that while the discussion above and FIG. 8 both refer to a linear relationship between DPOP and PA, a non-linear relationship between DPOP and PA may be used in some embodiments to correct DPOP. For example, in some embodiments it may be determined that the relationship between DPOP and PA can be expressed by the polynomial equation:

DPOP=m₁*PA²+m₂*PA+A (17)

In such a case, a non-linear correction may be applied using the following equation:

DPOP_{C}=DPOP_{M}−(PA_{M}²−PA_{C}²)*m₁−(PA_{M}−PA_{C})*m₂ (18)

It will be understood that in the case of eq. (18), (PA_{M}²−PA_{C}²)*m₁+(PA_{M}−PA_{C})*m₂, may be referred to as a correction factor. It will also be understood that in some embodiments, a higher order polynomial equation or any other suitable non-linear equation may be used to express the relationship between DPOP and PA, and appropriate correction may be applied based on the equation, the measured DPOP value, the measured PA value, and the standard PA value similar to that described above. For example, any suitable non-parametric and/or heteroassociative mapping may be used to create corrective formulas as alternatives to Eqs. (16) and (18) above, each with different correction factors used to correct the measured DPOP value. It will be understood that correction of DPOP or other fluid responsiveness parameters based on any of the PA or related parameters may be particularly useful for ventilated subjects under general anesthesia under full ventilator control, but may also be useful in the case of actively breathing subjects as well, including those under proportional ventilator control.

While the corrections described above with respect to eqs. (3)-(18) and FIGS. 5-8 are described as taking into account certain respiratory parameters individually, it will be understood that in some embodiments, any combination of respiratory parameters may be taken into account in correcting DPOP. In some embodiments, different respiratory parameters will be found to affect DPOP independently from other respiratory parameters, such that each of the corrections based on individual respiratory parameters may be performed independently to accurately correct DPOP. In other embodiments, different respiratory parameters may be interrelated such that it will be beneficial to determine the relationship between DPOP and the combination of respiratory parameters, and determine correction factors based on this combined relationship.

In some embodiments DPOP or other fluid responsiveness parameters may be corrected based on the error between DPOP and PPV as any of the above respiratory parameters changes. In some embodiments, the error between DPOP and PPV may be measured as the respiratory parameter changes. For example, the percent error between DPOP values and PPV values may be measured for different respiration rates. In some embodiments, the DPOP value may be corrected based on the mean error between DPOP and PPV. For example, DPOP may be corrected by subtracting the mean error between DPOP and PPV from the measured DPOP value. In some embodiments, the DPOP value may be corrected by determining the relationship between the DPOP/PPV error and the respiratory parameter, and using the relationship to determine a correction factor. For example, the error between DPOP values and PPV values may be plotted against respiration rate and a best fit line may be determined using statistical analysis such as regression analysis. The best fit line may then be used to correct DPOP based on the respiration rate. In some embodiments, the relationship between the DPOP/PPV error and the respiratory parameter may be evaluated piecewise. For example, various best fit lines may be determined for different ranges of the respiratory parameter, and these various relationships may be used to correct DPOP at the different ranges of the respiratory parameter.

It will be understood that while the above-mentioned corrections are discussed primarily in terms of DPOP, in some embodiments similar corrections may be applied to other fluid responsiveness parameters. For example, the typical threshold for determining that a patient is fluid responsive for a parameter such as PPV is approximately 15%. It will be understood that this threshold in the case of a spontaneously breathing subject may vary with respiratory parameters such as RR. Accordingly, in some embodiments, the PPV values may be corrected based on the relationship between PPV and RR similar to the methods described above. For example, the PPV values may be corrected so that the threshold for fluid responsiveness remains at approximately 15% regardless of RR.

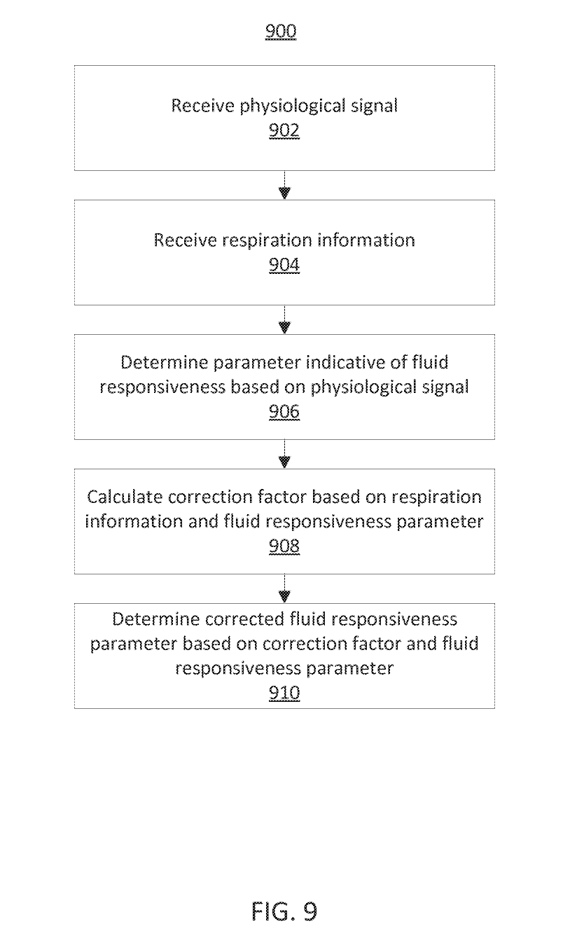

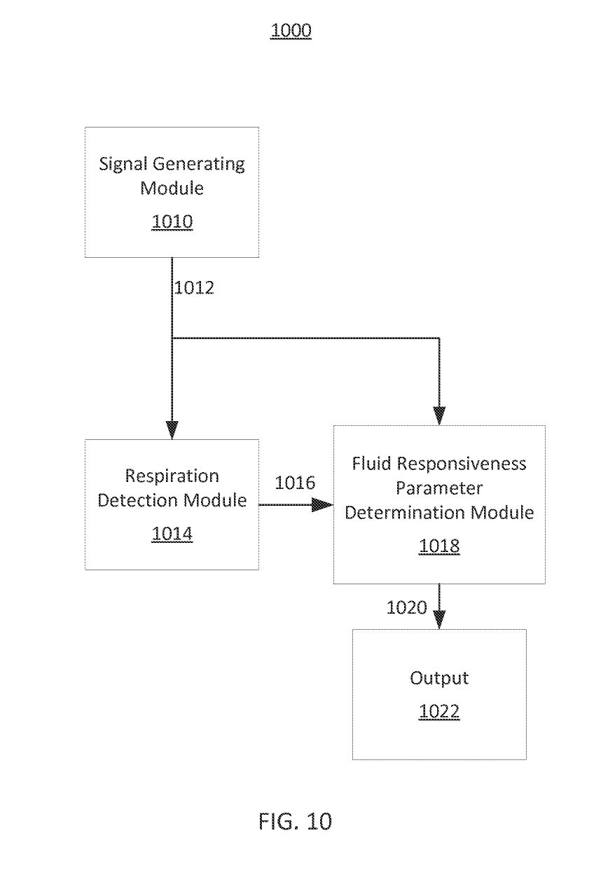

Determination of fluid responsiveness while correcting for respiration information in accordance with the present disclosure will be discussed with reference to FIGS. 9-10 below.

FIG. 9 shows illustrative steps 900 for determining fluid responsiveness in accordance with some embodiments of the present disclosure. Although exemplary steps are described herein, it will be understood that steps may be omitted and that any suitable additional steps may be added for determining respiration information. Although the steps described herein may be performed by any suitable device or system, in an exemplary embodiment, the steps may be performed by monitoring system 310, monitoring system 100, any components and modules thereof, and any combination thereof.

At step 902, the physiological monitoring system may receive a physiological signal. The physiological signal may be indicative of light attenuated by a subject. For example, the physiological signal may be a PPG signal received from a pulse oximeter.

At step 904, the physiological monitoring system may receive respiration information of a subject. In some embodiments, the respiration information may be at least one of a respiration rate of the subject, a respiration effort of the subject, a tidal volume of the subject, an airway or other respiratory pressure of the subject, any other respiration information of the subject, and any suitable combination thereof. In some embodiments, a module associated with, part of, or coupled to the physiological monitoring system may determine respiration information based on the physiological signal received in step 902 and may transmit this respiration information to the physiological monitoring system. For example, any suitable module may determine respiration information based on the physiological signal received in step 902 in accordance with embodiments disclosed in, U.S. patent application Ser. No. 13/243,785, filed on Sep. 23, 2011, the contents of which are entirely incorporated by reference herein. In some embodiments, respiration information may be received by the physiological monitoring system from an external device or monitoring system that measures such respiration information. For example, the respiration information may be received from a capnograph, a pulse oximeter, a trans-thoracic impedance device, a pneumotachometer, an assisted breathing device such as a ventilator, any other suitable source, and/or any suitable combination thereof.

At step 906 the system may determine a parameter indicative of fluid responsiveness based on the physiological signal received in step 902. The parameter indicative of fluid responsiveness may be determined in accordance with any of the above-mentioned methods. In some embodiments, the parameter indicative of fluid responsiveness may be determined by determining a plurality of amplitudes in the physiological signal and by identifying maximum and minimum amplitudes during a time window and dividing a difference between the amplitudes by an average of the amplitudes. For example the fluid responsiveness parameter may be determined based on equations (1)-(2) used to calculate DPOP as described above.

At step 908 a correction factor may be calculated based on the respiration information determined or received in step 904 and the fluid responsiveness parameter determined in step 906. In some embodiments, the correction factor may be calculated based on any of equations (3)-(18) discussed above or any other suitable equation derived from the relationship between the fluid responsiveness parameter and the respiration information of the subject. In some embodiments, the correction factor may be calculated by comparing the measured respiration rate to a standard respiration rate. For example, the measured respiration rate may be compared to a respiration rate typically used for a ventilated patient, such as 12 breaths per minute, and the correction factor may be determined based on the comparison and based on the relationship between the respiration rate and the fluid responsiveness parameter.

At step 910, a corrected fluid responsiveness parameter may be determined based on the fluid responsiveness parameter determined in step 906 and the correction factor calculated in step 908. In some embodiments, the corrected fluid responsiveness parameter may be determined based on any of equations (3)-(18) discussed above or any other suitable equation.

An illustrative physiological monitor 1000 for monitoring fluid responsiveness of a subject is shown in FIG. 10. The monitor 1000 includes a signal generating module 1010. In some embodiments, signal generating module 1010 may include any suitable combination of components of monitor 104 as described with respect to FIG. 1 for generating a physiological signal. For example, signal generating module 1010 may include light drive circuitry 120, control circuitry 110, and front end processing circuitry 150 as described above with respect to FIG. 1, and may be configured to generate signals and process them as described above. In some embodiments, signal generating module 1010 may include fewer components or additional components (e.g., sensor 102). Signal generating module 1010 may include one or more adjustable gains and generate a physiological signal. In some embodiments, the physiological signal may be a signal indicative of light attenuated by a subject. For example, the physiological signal may be a PPG signal generated by a pulse oximeter as described above with respect to FIGS. 1-3. In some embodiments, instead of signal generating module 1010, physiological monitor 1000 may include an input (not shown) configured to receive a physiological signal such as that described above as generated by signal generating module 1010. The input may be configured to receive a physiological signal from an external device configured to generate the physiological signal as described above.

Signal generating module 1010 generates output 1012. Output 1012 may include the physiological signal. In some embodiments, output 1012 is passed to respiration detection module 1014. In some embodiments, respiration detection module 1014 may be configured to determine respiration information based on output 1012. For example, respiration detection module 1014 may determine one or more of respiration rate, respiration effort, tidal volume, and airway pressure of a subject. In some embodiments, respiration detection module 1014 may include any suitable combination of components of monitor 104 as described with respect to FIG. 1 for analyzing and processing a physiological signal. For example, respiration detection module 1014 may include front end processing circuitry 150, back end processing circuitry 170, any components thereof, and/or any suitable combination thereof as described above with respect to FIG. 1, and may be configured to receive signals and process them as described above. In some embodiments, respiration detection module 1014 may include fewer components or additional components. Respiration detection module 1014 generates output 1016 that is passed to fluid responsiveness parameter determination module 1018. Output 1016 may include respiration information such as respiration rate, respiration effort, tidal volume, airway pressure, and/or any suitable combination thereof. In some embodiments, instead of respiration detection module 1014, physiological monitor 1000 may include an input (not shown) configured to receive any of the respiration information described herein. The input may be configured to receive any of the respiration information described herein from an external device such as a ventilator, a capnograph, a pulse oximeter, a trans-thoracic impedance device, or a pneumotachometer. In some embodiments, the input may be a part of any of signal generating module 1010, respiration module 1014, or fluid responsiveness parameter determination module 1018.

In some embodiments, output 1012 of signal generating module 1010 may also be passed to fluid responsiveness parameter determination module 1018. In some embodiments, fluid responsiveness parameter determination module 1018 may include any suitable combination of components of monitor 104 as described with respect to FIG. 1 for analyzing and processing a physiological signal. For example, fluid responsiveness parameter determination module 1018 may include front end processing circuitry 150, back end processing circuitry 170, any components thereof, and/or any suitable combination thereof as described above with respect to FIG. 1, and may be configured to receive signals and process them as described above. In some embodiments, fluid responsiveness parameter determination module 1018 may include fewer components or additional components. Fluid responsiveness parameter determination module 1018 may be configured to determine fluid responsiveness of a subject in accordance with any of the techniques described in the present disclosure. For example, fluid responsiveness parameter determination module 1018 may repeatedly calculate fluid responsiveness values such as DPOP based on Eqs. (1)-(2) discussed above over a calculation window. In some embodiments, the calculation window may be adjusted based on respiration information received from respiration detection module, such as respiration rate. In some embodiments, fluid responsiveness parameter determination module 1018 may take an average of the values determined over a second fixed calculation window.

In some embodiments, output 1012 of signal generating module 1010 may be passed to a filter module (not shown) prior to being passed to fluid responsiveness parameter determination module 1018. In some embodiments, the filter module may be configured to filter the physiological signal to generate a filtered signal which is passed to the fluid responsiveness parameter instead of or in addition to the physiological signal. In some embodiments, the filter module may filter the physiological signal by applying a band-pass filter on the physiological signal with thresholds set to remove non-respiration induced modulations in the physiological signal. In some embodiments, the filter module may include any suitable combination of components of monitor 104 as described with respect to FIG. 1 for analyzing and processing a physiological signal. For example, the filter module may include front end processing circuitry 150, back end processing circuitry 170, any components thereof, and/or any suitable combination thereof as described above with respect to FIG. 1, and may be configured to receive signals and process them as described above. In some embodiments, the filter module may include fewer components or additional components.

In some embodiments, fluid responsiveness parameter determination module 1018 may correct the fluid responsiveness values in accordance with any of the techniques described in the present disclosure. In some embodiments, the fluid responsiveness parameter determination module 1018 may correct the fluid responsiveness values based on respiration information generated by respiration detection module 1014 (or received from an input) as described with respect to FIGS. 5-8 and steps 908 and 910 of FIG. 9 above. In some embodiments, the fluid responsiveness parameter determination module 1018 may determine the relationship between any of the aforementioned respiration information and DPOP, or may reference a previous determination thereof stored in its memory, and determine a correction factor based on this relationship, the previously calculated DPOP, the calculated (or received) respiration information, and a standard associated with the respiration information. For example, the fluid responsiveness parameter determination module may reference a previously determined linear relationship between respiration rate of a subject and DPOP, and using the determined relationship and Eqs. (3) and (4), may correct the DPOP value as described above. For example, the fluid responsiveness parameter determination module may correct the DPOP value to reflect the DPOP value that would have been calculated if the respiration rate were that of a standard ventilation respiration rate, such as 12 breaths per minute. In some embodiments, a non-linear relationship between the respiration information and DPOP may be referenced and/or determined by fluid responsiveness parameter determination module 1018. In some embodiments, fluid responsiveness parameter determination module 1018 may determine what type of correction to be used, e.g. linear or non-linear, based on the measured DPOP and respiration information. For example, fluid responsiveness parameter determination module 1018 may monitor the measured DPOP and respiration rate over time and perform statistical analysis on the measured DPOP and respiration rate such as a regression analysis to determine that the relationship is sufficiently linear to use a linear correction to correct DPOP. In some embodiments, the fluid responsiveness parameter determination module 1018 may compare a result of the regression analysis to a threshold to determine whether to use a linear or non-linear correction to correct DPOP.

In some embodiments, fluid responsiveness parameter determination module 1018 may determine a corrected parameter indicative of fluid responsiveness 1020 in accordance with any of the above-mentioned techniques, including those discussed above with respect to FIGS. 5-9 and Eqs. 1-18, and pass it to output 1022.

Output 1022 may include display 184 and/or communication interface 190 of monitor 104 as described above with respect to FIG. 1, displays 320 and/or 328 of physiological monitoring system 310 as described above with respect to FIG. 3, any other suitable output, or any other suitable combination thereof. For example, the corrected parameter indicative of fluid responsiveness may be output to be displayed on display 320, display 328, display 184, or may be output to another device via communication interface 190, so that a clinician may diagnose a subject's condition and provide treatment in response thereto.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A physiological monitor for monitoring fluid responsiveness of a subject, comprising:
an input configured to receive a physiological signal; and
a fluid responsiveness parameter determination module configured to:
receive the physiological signal;
determine a parameter indicative of fluid responsiveness based on the physiological signal;
receive respiration information of the subject;
determine a correction factor of the parameter indicative of fluid responsiveness based on the respiration information; and
determine a corrected parameter indicative of fluid responsiveness based on the parameter indicative of fluid responsiveness and the correction factor.

2. The monitor of claim 1, wherein the respiration information comprises at least one of respiration rate, respiration effort, tidal volume, and airway pressure of the subject.

3. The monitor of claim 1, wherein the fluid responsiveness parameter determination module is further configured to:
compare the respiration information to a standard value; and
determine the correction factor based on the comparison.

4. The monitor of claim 3, wherein the respiration information comprises respiration rate, and wherein the standard value comprises a respiration rate typically used for a ventilated subject.

5. The monitor of claim 4, wherein the standard value comprises approximately 12 breaths per minute.

6. The monitor of claim 1, wherein the correction factor is determined using a type of correction selected from the group consisting of a linear correction, a non-linear correction, and a non-parametric correction.

7. The monitor of claim 6, wherein the type of correction is selected based on at least one of the respiration information and the parameter indicative of fluid responsiveness.

8. The monitor of claim 1, wherein the correction factor is determined using a neural network.

9. The monitor of claim 1, further comprising:
a filter module configured to adjustably filter the physiological signal based on the respiration information to generate a filtered signal; and
wherein the fluid responsiveness parameter determination module is further configured to determine the parameter indicative of fluid responsiveness based on the filtered signal.

10. The monitor of claim 1, wherein the subject is not on a ventilator.

11. The monitor of claim 1, wherein the respiration information is received from a ventilator.

12. A physiological monitor for monitoring fluid responsiveness of a subject, comprising:
a signal generating module configured to generate a physiological signal that is indicative of light attenuated by a subject;
a respiration detection module configured to determine a respiration rate of the subject; and
a fluid responsiveness parameter determination module configured to determine a parameter indicative of fluid responsiveness based on respiratory modulations in the physiological signal and the respiration rate of the subject.

13. The monitor of claim 12, wherein the fluid responsiveness parameter determination module is further configured to:
determine an initial parameter indicative of fluid responsiveness based on respiratory modulations in the physiological signal;
compare the respiration rate to a standard respiration rate; and
normalize the initial parameter indicative of fluid responsiveness based on the comparison to determine the parameter indicative of fluid responsiveness.

14. The monitor of claim 13, wherein the standard respiration rate comprises a respiration rate typically used for a ventilated subject.

15. The monitor of claim 13, wherein the standard respiration rate is approximately 12 breaths per minute.

16. The monitor of claim 12, wherein the fluid responsiveness parameter determination module is further configured to:
determine an initial parameter indicative of fluid responsiveness based on respiratory modulations in the physiological signal;
compare the respiration rate to a standard respiration rate; and
correct the initial parameter indicative of fluid responsiveness based on the comparison to determine the parameter indicative of fluid responsiveness using a type of correction selected from the group consisting of a linear correction, a non-linear correction, and a non-parametric correction.

17. The monitor of claim 12, wherein the parameter indicative of fluid responsiveness is determined using a neural network.

18. The monitor of claim 12, wherein the fluid responsiveness parameter determination module is further configured to:
determine a maximum amplitude in the physiological signal;
determine a minimum amplitude in the physiological signal; and
determine the parameter indicative of fluid responsiveness by dividing a difference between the maximum and minimum amplitudes by an average of the maximum and minimum amplitudes.

19. A method for determining fluid responsiveness in a subject comprising:
receiving a physiological signal;
determining respiration information of the subject based on the physiological signal;
determining a plurality of amplitudes in the physiological signal;
determining a parameter indicative of fluid responsiveness of the subject based on the plurality of amplitudes;
calculating a correction factor based on the respiration information and the parameter indicative of fluid responsiveness of the subject; and
determining a corrected parameter indicative of fluid responsiveness of the subject based on the correction factor and the parameter indicative of fluid responsiveness of the subject.

20. The method of claim 19, wherein:
the respiration information comprises respiration rate;
determining the parameter indicative of fluid responsiveness comprises dividing a difference between a maximum amplitude and a minimum amplitude by an average of the maximum and minimum amplitudes; and
calculating the correction factor comprises:
comparing the respiration rate to a standard respiration rate; and
calculating the correction factor based on the comparison.
